(19) United States
(12) Patent Application Publication
(10) Pub. No.: US 2024/0325266 A1
(43) Pub. Date: Oct. 3, 2024

(54) Personal Care Compositions

(71) Applicant: Colgate-Palmolive Company, New York, NY (US)

(72) Inventors: Carolina BERMUDEZ SALGUERO, Mexico City (MX); Luis Alberto CRUZ, Mexico City (MX); Edgar H. HERNANDEZ, Mexico City (MX); Juan Mauricio MORA-PALE, Mexico City (MX); Sarita Vera MELLO, Ringwood, NJ (US)

(73) Assignee: Colgate-Palmolive Company, New York, NY (US)

(21) Appl. No.: 18/621,283

(22) Filed: Mar. 29, 2024

Related U.S. Application Data
(60) Provisional application No. 63/493,404, filed on Mar. 31, 2023.

(51) Int. Cl. A61K 8/34; A61K 8/02; A61Q 15/00

(52) U.S. Cl. CPC A61K 8/345; A61Q 15/00; A61K 8/0216; A61K 2800/30; A61K 2800/874

(57) ABSTRACT

Described herein are personal care compositions comprising a liquid carrier; alkyl glyceryl ether; xanthan gum; an ester of glycerin and a C₈₋₁₈ fatty acid; polyol; and an alkali salt of a C₁₄₋₂₂ fatty acid.

### CROSS-REFERENCE TO RELATED APPLICATIONS

This application claims the benefit of priority from U.S. Patent Application No. 63/493,404, filed Mar. 31, 2023, titled Personal Care Compositions, the content of which are hereby incorporated herein in their entireties, for all purposes.

### BACKGROUND

Numerous underarm products contain metal-salts, such as aluminum salts, to impart malodor and/or antiperspirant protection. However, consumer preference for metal-salt free products has increased over the years. There is a trend for individuals to select deodorant compositions which are metal-salt free. Therefore, the need exists for new deodorant formulations that provide protection against malodor and sweat without the presence of such metal salts.

### BRIEF SUMMARY

The present invention is directed to a deodorant composition comprising a liquid carrier; alkyl glyceryl ether; xanthan gum; an ester of glycerin and a C₈₋₁₈ fatty acid; polyol; and an alkali salt of a C₁₄₋₂₂ fatty acid.

Other embodiments of the present invention include a deodorant composition comprising: a liquid carrier; methylheptylglycerin; xanthan gum; glyceryl monolaurate; a polyol comprising a diol and a triol; and sodium stearate.

Other embodiments of the present invention include a deodorant composition comprising: a liquid carrier; a polyol; methylheptylglycerin in an amount ranging from about 0.5 wt. % to about 2.5 wt. % based on the total weight of the deodorant composition; xanthan gum in an amount ranging from about 0.1 wt. % to about 0.6 wt. % based on the total weight of the deodorant composition; sodium stearate in an amount ranging from about 1 wt. % to about 12 wt. % based on the total weight of the deodorant composition; and glyceryl monolaurate in an amount ranging from about 0.25 wt. % to about 2.25 wt. % based on the total weight of the deodorant composition.

Other embodiments of the present invention include a method of preventing malodor comprising applying one or more of the aforementioned deodorant compositions to a skin surface.

Other embodiments of the present invention include a method of manufacturing a deodorant composition comprising: a) mixing a liquid carrier, methylheptylglycerin, xanthan gum, sodium stearate, polyol or polyols, and glyceryl monolaurate to form a blend; b) adding a predetermined quantity of the blend to a container.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

A list of non-limiting, example embodiments is provided below.

In accordance with embodiment 1, provided is a personal care composition comprising:
optionally, a liquid carrier;
alkyl glyceryl ether;
xanthan gum;
an ester of glycerin and a C8-18 fatty acid;
polyol comprising glycerin; and
an alkali salt of a C14-22 fatty acid.

In accordance with embodiment 2, provided a personal care composition according to embodiment 1, wherein the alkyl glyceryl ether comprises methylheptylglycerin and is present in an amount ranging from about 0.5 wt. % to about 2.5 wt. %, based on the total weight of the deodorant composition.

In accordance with embodiment 3, provided a personal care composition according to any one of embodiments 1 to 2, wherein the xanthan gum is present in an amount ranging from about 0.1 wt. % to about 0.6 wt. %, based on the total weight of the deodorant composition.

In accordance with embodiment 4, provided a personal care composition according to any one of embodiments 1 to 3, wherein the ester of glycerin and a C8-18 fatty acid is glyceryl monolaurate and is present in an amount ranging from about 0.25 wt. % to about 2.25 wt. %, based on the total weight of the deodorant composition.

In accordance with embodiment 5, provided a personal care composition according to anyone of embodiments 1 to 4, wherein the polyol comprises a diol and a triol.

In accordance with embodiment 6, provided a personal care composition according to embodiment 5, wherein the diol is selected from one or more of propylene glycol, butylene glycol, and combinations thereof, and is present in an amount ranging from about 40 wt. % to about 66 wt. %, based on the total weight of the deodorant composition.

In accordance with embodiment 7, provided a personal care composition according to anyone of embodiments 5 to 6, wherein the triol comprises glycerin and is present in an amount ranging from about 1 wt. % to about 20 wt. %, based on the total weight of the deodorant composition.

In accordance with embodiment 8, provided a personal care composition according to any one of embodiments 1 to 7, wherein the alkali salt of the C14-22 fatty acid comprises sodium stearate in an amount ranging from about 1 wt. % to about 12 wt. %, based on the total weight of the deodorant composition.

In accordance with embodiment 9, provided a personal care composition according to any one of embodiments 1 to 8, wherein the liquid carrier comprises water, and the liquid carrier is present in an amount ranging from about 15 wt. % to about 35 wt. %, based on the total weight of the deodorant composition.

In accordance with embodiment 10, provided a personal care composition according to any one of embodiments 1 to 9, wherein the deodorant composition is in a solid state at room temperature.

In accordance with embodiment 11, provided a personal care composition comprising:
optionally, a liquid carrier;
methylheptylglycerin;
xanthan gum;
glyceryl monolaurate;
a polyol comprising a diol and a triol; and
sodium stearate.

In accordance with embodiment 12, provided a personal care composition according to embodiment 11, wherein the methylheptylglycerin is present in an amount ranging from about 0.5 wt. % to about 2.5 wt. %, based on the total weight of the deodorant composition.

In accordance with embodiment 13, provided a personal care composition according to any one of embodiments 11 to 12, wherein the xanthan gum is present in an amount ranging from about 0.1 wt. % to about 0.6 wt. %, based on the total weight of the deodorant composition.

In accordance with embodiment 14, provided a personal care composition according to any one of embodiments 11 to 13, wherein the glyceryl monolaurate is present in an amount ranging from about 0.25 wt. % to about 2.25 wt. %, based on the total weight of the deodorant composition.

In accordance with embodiment 15, provided a personal care composition according to anyone of embodiments 11 to 14, wherein the diol is present in an amount ranging from about 40 wt. % to about 66 wt. %, based on the total weight of the deodorant composition.

In accordance with embodiment 16, provided a personal care composition according to embodiment 15, wherein the diol is selected from one or more of propylene glycol, butylene glycol, and combinations thereof.

In accordance with embodiment 17, provided a personal care composition according to any one of embodiments 11 to 16, wherein the triol comprises glycerin in an amount ranging from about 1 wt. % to about 20 wt. %, based on the total weight of the deodorant composition.

In accordance with embodiment 18, provided a personal care composition according to any one of embodiments 11 to 17, wherein the sodium stearate is present in an amount ranging from about 1 wt. % to about 12 wt. %, based on the total weight of the deodorant composition.

In accordance with embodiment 19, provided a personal care composition according to any one of embodiments 11 to 18, wherein the liquid carrier comprises water, and the liquid carrier is present in an amount ranging from about 15 wt. % to about 35 wt. %, based on the total weight of the deodorant composition.

In accordance with embodiment 20, provided a personal care composition according to any one of embodiments 11 to 18, wherein the deodorant composition is in a solid state at room temperature.

In accordance with embodiment 21, provided a personal care composition comprising:
a polyol (preferably, comprising glycerin);
methylheptylglycerin in an amount ranging from about 0.5 wt. % to about 2.5 wt. %, based on the total weight of the deodorant composition;
xanthan gum in an amount ranging from about 0.1 wt. % to about 0.6 wt. %, based on the total weight of the deodorant composition;
sodium stearate in an amount ranging from about 1 wt. % to about 12 wt. %, based on the total weight of the deodorant composition; and
glyceryl monolaurate in an amount ranging from about 0.25 wt. % to about 2.25 wt. %, based on the total weight of the deodorant composition,
wherein the personal care composition is in the form of a deodorant composition.

In accordance with embodiment 22, provided a personal care composition according to embodiment 21, wherein the polyol selected from one or more of glycerin, propylene glycol, butylene glycol, and combinations thereof.

In accordance with embodiment 23, provided a personal care composition according to embodiment 22, wherein the polyol is present in an amount ranging from about 51 wt. % to about 81 wt. %, based on the total weight of the deodorant composition.

In accordance with embodiment 24, provided a personal care composition according to any one of embodiments 21 to 23, wherein the liquid carrier comprises water, and the liquid carrier is present in an amount ranging from about 15 wt. % to about 35 wt. %, based on the total weight of the deodorant composition.

In accordance with embodiment 25, provided a personal care composition according to any one of embodiments 21 to 24, wherein the deodorant composition is in a solid state at room temperature.

In accordance with embodiment 26, provided a personal care composition according to embodiment 25, wherein the deodorant composition in the solid state is formed into a predetermined shape.

In accordance with embodiment 27, provided a personal care composition according to any foregoing embodiment, wherein the personal care composition reduces sweat by at least 21%, measured 1 hour after application of the personal care composition.

In accordance with embodiment 28, provided a personal care composition according to any foregoing embodiment, wherein the personal care composition reduces sweat by at least 9%, measured 24 hours after application of the personal care composition.

In accordance with embodiment 29, provided is a method of reducing and/or preventing malodor comprising applying one or more of the personal care compositions according to embodiments 1 to 28 to a skin surface.

In accordance with embodiment 30, provided a method according to embodiment 29, wherein the deodorant is applied by sliding the personal care composition to the skin surface.

In accordance with embodiment 31, provided a method according embodiment 29 or embodiment 30, wherein the personal care is in a solid state when applied to the skin surface.

In accordance with embodiment 32, provided a method according to any one of embodiments 29 to 31, wherein the personal care is selected from: a stick-type deodorant; a stick-type antiperspirant; a roll-on type deodorant; a roll-on type antiperspirant; a lotion; a cream; and a balm.

In accordance with embodiment 33, provided a method of manufacturing a personal care composition comprising:
a) mixing a liquid carrier, methylheptylglycerin, xanthan gum, sodium stearate, and glyceryl monolaurate to form a blend;
b) adding a predetermined quantity of the blend to a container; and
c) cooling the blend in the container to room temperature to form a solid state.

In accordance with embodiment 34, provided a method according to embodiment 33, wherein the liquid carrier comprises water, and the liquid carrier is present in an amount ranging from about 15 wt. % to about 35 wt. %, based on the total weight of the blend.

In accordance with embodiment 35, provided a method according to embodiment 33 or embodiment 34, wherein the methylheptylglycerin is present in an amount ranging from about 0.5 wt. % to about 2.5 wt. %, based on the total weight of the blend.

In accordance with embodiment 36, provided a method according to any one of embodiments 33 to 35, wherein the xanthan gum is present in an amount ranging from about 0.1 wt. % to about 0.6 wt. %, based on the total weight of the blend.

In accordance with embodiment 37, provided a method according to any one of embodiments 33 to 36, wherein sodium stearate is present in an amount ranging from about 1 wt. % to about 12 wt. %, based on the total weight of the blend.

In accordance with embodiment 38, provided a method according to any one of embodiments 33 to 37, wherein the glyceryl monolaurate is present in an amount ranging from about 0.25 wt. % to about 2.25 wt. % based on the total weight of the blend.

In accordance with embodiment 39, provided a method according to embodiment 38, wherein the diol is present in an amount ranging from about 40 wt. % to about 66 wt. %, based on the total weight of the blend.

In accordance with embodiment 40, provided a method according to any one of embodiments 33 to 39, wherein the mixing of step a) is performed at a temperature ranging from about 75° F. to about 185° F.

In accordance with embodiment 41, provided a method according to any one of embodiments 33 to 40, wherein step b) is performed at an elevated temperature and the blend is in a liquid or semi-liquid state.

In accordance with embodiment 42, provided a method according to embodiment 41, wherein the method further comprises step c), wherein the blend is cooled to room temperature to form a solid state.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention.

Unless otherwise specified, all percentages and amounts expressed herein and elsewhere in the specification should be understood to refer to percentages by weight. The amounts given are based on the active weight of the material. According to the present application, the term “about” means+/−10% of the reference value. According to the present application, the term “substantially free” is less than about 0.1 wt. % based on the total of the referenced value.

As used herein, the use of a compound comprising several isomers or stereoisomers includes all the isomeric forms of that compound. When referring to chemical structures, and names, the symbols “C”, “H”, and “O” mean carbon, hydrogen, and oxygen, respectively. The symbols “-”, “=” and “=” mean single bond, double bond, and triple bond, respectively. For readability purposes, the chemical functional groups are in their adjective form; for each of the adjectives, the word “group” is assumed. For example, the adjective “alkyl” without a noun thereafter, should be read as “an alkyl group.”

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, caprylyl glycol may be characterized as both a humectant and a preservative. If a particular personal care composition includes both a humectant and a preservative, caprylyl glycol will serve only as either a humectant or a preservative—not both.

As used herein, the terms “composition” and “formulation” can be used interchangeably within the specification. As used herein, the term “antiperspirant” refers to a product applied topically that reduces the production of perspiration (sweat) at that site. As used herein the term “reduce” in the “reduce sweat” context, refers to decrease or lessen of the dampness, perspiration, or wetness in the applied area. As used herein, the term “deodorant” refers to inhibiting and/or reducing the growth of the odor causing bacteria that makes the sweat smell. As used herein, the term “antiperspirant/deodorant compositions” refers to compositions which provide an antiperspirant effect and/or a deodorant effect.

As used herein, the term “free of aluminum-based antiperspirant actives”, means that the antiperspirant/deodorant compositions of the present disclosure do not comprise aluminum-based antiperspirant actives. For example, aluminum-based antiperspirant actives are not added to the personal care compositions of the invention to display some personal care effect.

As used herein, the term “aluminum free” or “free-from aluminum” means that the antiperspirant/deodorant composition does not contain any aluminum-based antiperspirant. Non limiting examples of aluminum-based antiperspirant actives, can include those listed in the US antiperspirant monograph, such as, aluminum chlorohydrate, aluminum chloride, aluminum dichlorohydrate, aluminum sesquichlorohydrate poylethylenc glycol, aluminum sesquichlorohydrate propylene glycol, aluminum zirconium octachlorohydrate, aluminum zirconium pentachlorohydrate, aluminum zirconium tetrachlorohydrate, aluminum zirconium trichlorohydrate, aluminum sesquichlorohydrate, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex propylene glycol (aluminum chlorohydrex PG), aluminum chlorohydrex polyethylene glycol (aluminum chlorohydrex PEG), aluminum dichlorohydrex propylene glycol (aluminum dichlorohydrex PG), and aluminum dichlorohydrex polyethylene glycol (aluminum dichlorohydrex PEG). In some embodiments, the personal care composition may be free of one or more of the foregoing examples of aluminum-based antiperspirant actives.

As used herein, the term “zirconium free” or “free-from zirconium-based antiperspirant actives” means that the antiperspirant/deodorant composition does not contain any zirconium-based antiperspirant. Non limiting examples of zirconium-based antiperspirant actives, include aluminum zirconium octachlorohydrate, aluminum zirconium pentachlorohydrate, aluminum zirconium tetrachlorohydrate, aluminum zirconium trichlorohydrate, and aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly). In some embodiments, the personal care composition may be free of one or more of the foregoing examples of zirconium-based antiperspirant actives.

As used herein, the term “free of zinc-based antiperspirant actives”, means that the antiperspirant/deodorant composition does not comprise zinc-based antiperspirant actives. Zinc-based antiperspirant actives are not added to the antiperspirant/deodorant compositions of the invention to display some antiperspirant/deodorant effect. Non limiting examples of zinc-based antiperspirant actives can include one or more of zinc oxide, zinc hydroxide, zinc hydroxide ions with counter ions, and zinc ions with counter ions. In some embodiments, the personal care composition may be free of one or more of the foregoing examples of zinc-based antiperspirant actives.

As used herein, the term “free of iron-based antiperspirant actives”, means that the antiperspirant/deodorant composition does not comprise one or more of iron-based antiperspirant active.

As used herein, the term “free of titanium-based antiperspirant actives”, means that the antiperspirant/deodorant composition does not comprise titanium-based antiperspirant actives. For example, the antiperspirant/deodorant compositions may include a nominal amount, be substantially free of, or free of titanium-based antiperspirant actives, such as ammonium titanium lactate and the like. In some embodiments, the personal care composition may be free of one or more of the foregoing examples of titanium-based antiperspirant actives.

As used herein, the term “free of magnesium-based antiperspirant actives”, means that the antiperspirant/deodorant composition does not comprise magnesium-based antiperspirant actives. Non-limiting examples of magnesium-based actives include, but are not limited to, magnesium chloride, magnesium bromide, magnesium fluoride and organic salts such as various alkyl chain length substituted carboxylic acids, magnesium oxide, and magnesium hydroxide. As used herein, the term “axilla” or “axillary region” refers to the armpit or underarm of the body. In some embodiments, the personal care composition may be free of one or more of the foregoing examples of magnesium-based antiperspirant actives.

The antiperspirant/deodorant compositions may be substantially free or free of aluminum-based antiperspirant, magnesium-based antiperspirant actives, titanium-based antiperspirant actives, free-from zirconium-based antiperspirant actives, and zinc-based antiperspirant actives. For example, the antiperspirant/deodorant compositions may have about 3 wt. % or less, about 2 wt. % or less, about 1 wt. % or less, about 0.5 wt. % or less, about 0.1 wt. % or less, about 0.05 wt. % or less, or about 0.01 wt. % or less of aluminum-based antiperspirant, magnesium-based antiperspirant actives, titanium-based antiperspirant actives, zirconium-based antiperspirant actives, and zinc-based antiperspirant actives (individually or cumulatively), based on the total weight of the antiperspirant/deodorant composition. In some embodiments, the antiperspirant/deodorant compositions have about 0 wt. % or 0 wt. % of aluminum-based antiperspirant, magnesium-based antiperspirant actives, titanium-based antiperspirant actives, zirconium-based antiperspirant actives, and zinc-based antiperspirant actives (individually or cumulatively), based on the total weight of the antiperspirant/deodorant composition.

Aspects of the invention are directed to antiperspirant/deodorant compositions that provide a reduction of sweat while being free-from aluminum-based antiperspirant and, preferably, free of magnesium-based antiperspirant actives, titanium-based antiperspirant actives, zirconium-based antiperspirant actives, and zinc-based antiperspirant actives. The inventors discovered that certain embodiments of antiperspirant/deodorant compositions containing specific combinations of ingredients in particular amounts and ratios provides a reduction of sweat of about 5% or more, about 10% or more, about 15% or more, about 20% or more, about 25% or more, about 30% or more, about 35% or more, about 40% or more, or about 45% or more, while being free from free-from aluminum-based antiperspirant, magnesium-based antiperspirant actives, and zinc-based antiperspirant actives.

The antiperspirant/deodorant compositions as disclosed herein, may retard the sweating process by reducing the amount of perspiration excreted from the eccrine sweat glands and/or apocrine sweat glands. In certain embodiments, the antiperspirant/deodorant composition provides a reduction of sweat of about 20% or more, about 23% or more, about 26% or more, about 29% or more, about 32% or more, about 35% or more, about 36% or more, about 40% or more, about 44% or more, or about 48% or more, preferably, over a 24-hour period after application. For example, the antiperspirant/deodorant composition may provide a reduction of sweat (e.g., produce a reduction of sweat when applied to skin) of about 20 to about 48%, about 20 to about 45%, about 20 to about 40%, about 20 to about 36%, about 20 to about 32%, about 20 to about 28%; from about 24 to about 48%, about 24 to about 45%, about 24 to about 40%, about 24 to about 36%, about 24 to about 32%, about 24 to about 28%; from about 28 to about 48%, about 28 to about 45%, about 28 to about 40%, about 28 to about 36%, about 28 to about 32%; from about 32 to about 48%, about 32 to about 45%, about 32 to about 40%, about 32 to about 36%; from about 36 to about 48%, about 36 to about 45%, about 36 to about 40%, or any range or subrange thereof, preferably, over a 1 hour, 12 hour, 24 hour or 48 hour period after application, e.g., as compared to not utilizing the antiperspirant/deodorant composition. In some embodiments, the antiperspirant/deodorant compositions described herein provide a reduction of sweat according to any of the foregoing listed ranges and/or percentages over about a 1 hour period, a 2 hour period, a 3 hour period, a 4 hour period, a 6 hour period, an 8 hour period, a 12 hour period, an 18 hour period, a 24 hour period, 36 hour period, or a 48 hour period, e.g., as compared to not utilizing the antiperspirant/deodorant composition.

In some embodiments, the invention provides antiperspirant/deodorant compositions that inhibit and/or reduce the growth of detrimental bacteria, responsible for the underarm malodor. By reducing the underarm sweat, the antiperspirant and/or deodorant compositions will reduce the formation of the detrimental bacteria responsible for the malodor. According to at least one aspect, a method is provided for reducing sweat in the axillary region, e.g., by applying the antiperspirant/deodorant compositions to the axillary region. In accordance with another aspect, provided is a method of reducing malodor caused by excessive sweat using the antiperspirant/deodorant compositions according to the present invention.

The antiperspirant/deodorant compositions can be used in a method to reduce sweat by applying the antiperspirant/deodorant compositions to the skin. In certain embodiments, the application is to axilla. In other embodiments, the invention provides a method of reducing sweat from about 10 to about 40%, or from about 15 to about 20%, or from about 25 to about 30%, or from about 30 to about 35%, or from about 35 to about 40% for about a 1 hour period, a 2 hour period, a 3 hour period, a 4 hour period, a 6 hour period, an 8 hour period, a 12 hour period, an 18 hour period, a 24 hour period, 36 hour period, or a 48 hour period, e.g., as compared to not utilizing the antiperspirant/deodorant composition.

In some embodiments, the invention provides a method of reducing sweat of a subject in need thereof, wherein the sweat reduction is of at least 5%, at least 10%, at least 15%, at least 20%, at least 25%, at least 30%, at least 35%, at least 40%, or at least 45% for about a 1 hour period, a 2 hour period, a 3 hour period, a 4 hour period, a 6 hour period, an 8 hour period, a 12 hour period, an 18 hour period, a 24 hour period, 36 hour period, or a 48 hour period, e.g., as compared to not utilizing the antiperspirant/deodorant composition.

Suitable components, such as those listed below, may be included or excluded from the formulations for the antiperspirant/deodorant compositions care compositions depending on the specific combination of other components, the form of the hair care compositions, and/or the use of the formulation (e.g., a roll-on, stick, spray, cream, etc.).

The present invention is directed to a deodorant composition. In some embodiments, the deodorant composition may be an underarm stick-type deodorant. In some embodiments, the deodorant composition may be an underarm roll-on type deodorant. In some embodiments, the deodorant composition may be a spray or a cream (e.g., an underarm spray or cream). The deodorant composition may comprise one or more all-natural or naturally derived compounds.

The deodorant composition typically comprises one or more polyol(s), which may be selected from one or more humectant(s). The deodorant composition may comprise a polyol. In some embodiments, the polyol may comprise a diol. In some embodiments, the polyol may comprise a triol. In some embodiments, the polyol may comprise a diol and a triol.

The polyol may be present in an amount ranging from about 5 wt. % to about 85 wt. % based on the total weight of the deodorant composition—including all wt. % and sub-ranges there-between. In some embodiments, the polyol may be present in an amount ranging from about 10 wt. % to about 60 wt. % based on the total weight of the deodorant composition—including all wt. % and sub-ranges there-between. In some embodiments, the polyol may be present in an amount ranging from about 57 wt. % to about 75 wt. % based on the total weight of the deodorant composition—including all wt. % and sub-ranges there-between. In some embodiments, the polyol may be present in an amount ranging from about 60 wt. % to about 72 wt. % based on the total weight of the deodorant composition—including all wt. % and sub-ranges there-between. In some embodiments, the polyol may be present in an amount ranging from about 63 wt. % to about 69 wt. % based on the total weight of the deodorant composition—including all wt. % and sub-ranges there-between. In some embodiments, the polyol may be present in an amount ranging from about 65 wt. % to about 67 wt. % based on the total weight of the deodorant composition—including all wt. % and sub-ranges there-between.

The diol and the triol may be present in a weight ratio ranging from about 0.5:1 to about 15:1—including all ratios and sub-ranges there-between. In some embodiments, the diol and the triol may be present in a weight ratio ranging from about 1:1 to about 7:1—including all ratios and sub-ranges there-between. In some embodiments, the diol and the triol may be present in a weight ratio ranging from about 1:1 to about 6:1—including all ratios and sub-ranges there-between.

The diol may be a bio-based diol. The diol may comprise a C₃ to C₁₀ diol. In some embodiments, the diol may be selected from one or more of propane diol, butane diol, pentane diol, combinations thereof.

The propane diol may comprise one or more of 1,2-propanediol (also referred to as propylene glycol) and 1,3-propanediol. The butane diol may comprise one or more of 1,2-butanediol, 1,3-butanediol (also referred to as butylene glycol), 2,3-butanediol, 1,4-butane diol. The pentane diol may comprise one or more of 1,2-pentanediol, 1,3-pentanediol, 2,3-pentanediol, 1,5-pentanediol.

The diol may be present in an amount ranging from about 5 wt. % to about 70 wt. % based on the total weight of the deodorant composition—including all percentages and sub-ranges there-between. In some embodiments, the diol may be present in an amount ranging from about 10 wt. % to about 60 wt. % based on the total weight of the deodorant composition—including all percentages and sub-ranges there-between. In some embodiments, the diol may be present in an amount ranging from about 50 wt. % to about 62 wt. % based on the total weight of the deodorant composition—including all percentages and sub-ranges there-between. In some embodiments, the diol may be present in an amount ranging from about 52 wt. % to about 60 wt. % based on the total weight of the deodorant composition—including all percentages and sub-ranges there-between. In some embodiments, the diol may be present in an amount ranging from about 54 wt. % to about 58 wt. % based on the total weight of the deodorant composition—including all percentages and sub-ranges there-between. In some embodiments, the diol may be present in an amount ranging from about 56 wt. % to about 57 wt. % based on the total weight of the deodorant composition—including all percentages and sub-ranges there-between.

In some embodiments, the diol may comprise propylene glycol and may be present in an amount ranging from about 40 wt. % to about 66 wt. % based on the total weight of the deodorant composition—including all percentages and sub-ranges there-between. In some embodiments, the diol may comprise propylene glycol and may be present in an amount ranging from about 48 wt. % to about 64 wt. % based on the total weight of the deodorant composition—including all percentages and sub-ranges there-between. In some embodiments, the diol may comprise propylene glycol and may be present in an amount ranging from about 50 wt. % to about 62 wt. % based on the total weight of the deodorant composition—including all percentages and sub-ranges there-between. In some embodiments, the diol may comprise propylene glycol and may be present in an amount ranging from about 52 wt. % to about 60 wt. % based on the total weight of the deodorant composition—including all percentages and sub-ranges there-between. In some embodiments, the diol may comprise propylene glycol and may be present in an amount ranging from about 54 wt. % to about 58 wt. % based on the total weight of the deodorant composition-including all percentages and sub-ranges there-between. In some embodiments, the diol may comprise propylene glycol and may be present in an amount ranging from about 54 wt. % to about 58 wt. %, or about 55 wt. %, about 56 wt. %, about 57 wt. %, based on the total weight of the deodorant composition—including all percentages and sub-ranges there-between.

In some embodiments, the diol may comprise a blend of propylene glycol and butylene glycol whereby the propylene glycol may be present in an amount ranging from about 32 wt. % to about 52 wt. % and the butylene glycol may be present in an amount ranging from about 4 wt. % to about 24 wt. % based on the total weight of the deodorant composition—including all percentages and sub-ranges there-between. In some embodiments, the diol may comprise a blend of propylene glycol and butylene glycol whereby the propylene glycol may be present in an amount ranging from about 34 wt. % to about 50 wt. % and the butylene glycol may be present in an amount ranging from about 6 wt. % to about 22 wt. % based on the total weight of the deodorant composition—including all percentages and sub-ranges there-between. In some embodiments, the diol may comprise a blend of propylene glycol and butylene glycol whereby the propylene glycol may be present in an amount ranging from about 36 wt. % to about 48 wt. % and the butylene glycol may be present in an amount ranging from about 8 wt. % to about 20 wt. % based on the total weight of the deodorant composition—including all percentages and sub-ranges there-between. In some embodiments, the diol may comprise a blend of propylene glycol and butylene glycol whereby the propylene glycol may be present in an amount ranging from about 38 wt. % to about 46 wt. % and the butylene glycol may be present in an amount ranging from about 10 wt. % to about 18 wt. % based on the total weight of the deodorant composition—including all percentages and sub-ranges there-between. In some embodiments, the diol may comprise a blend of propylene glycol and butylene glycol whereby the propylene glycol may be present in an amount ranging from about 40 wt. % to about 44 wt. % and the butylene glycol may be present in an amount ranging from about 12 wt. % to about 16 wt. % based on the total weight of the deodorant composition—including all percentages and sub-ranges there-between. In some embodiments, the diol may comprise a blend of propylene glycol and butylene glycol whereby the propylene glycol may be present in an amount ranging from about 42 wt. % to about 43 wt. % and the butylene glycol may be present in an amount ranging from about 14 wt. % to about 15 wt. % based on the total weight of the deodorant composition—including all percentages and sub-ranges there-between.

The triol may be selected from glycerin. The triol may be present in an amount ranging from about 1 wt. % to about 20 wt. % based on the total weight of the deodorant composition-including all percentages and sub-ranges there-between. In some embodiments, the triol may be present in an amount ranging from about 2 wt. % to about 19 wt. % based on the total weight of the deodorant composition—including all percentages and sub-ranges there-between. In some embodiments, the triol may be present in an amount ranging from about 3 wt. % to about 18 wt. % based on the total weight of the deodorant composition—including all percentages and sub-ranges there-between. In some embodiments, the triol may be present in an amount ranging from about 4 wt. % to about 17 wt. % based on the total weight of the deodorant composition-including all percentages and sub-ranges there-between. In some embodiments, the triol may be present in an amount ranging from about 5 wt. % to about 16 wt. % based on the total weight of the deodorant composition—including all percentages and sub-ranges there-between. In some embodiments, the triol may be present in an amount ranging from about 6 wt. % to about 15 wt. % based on the total weight of the deodorant composition—including all percentages and sub-ranges there-between. In some embodiments, the triol may be present in an amount ranging from about 7 wt. % to about 14 wt. % based on the total weight of the deodorant composition—including all percentages and sub-ranges there-between. In some embodiments, the triol may be present in an amount ranging from about 8 wt. % to about 13 wt. % based on the total weight of the deodorant composition-including all percentages and sub-ranges there-between. In some embodiments, the triol may be present in an amount ranging from about 9 wt. % to about 12 wt. % based on the total weight of the deodorant composition—including all percentages and sub-ranges there-between. In some embodiments, the triol may be present in an amount ranging from about 10 wt. % to about 11 wt. % based on the total weight of the deodorant composition—including all percentages and sub-ranges there-between.

Without being limited to any particular theory, it was unexpectedly discovered that certain amounts of glycerin in combination with certain amounts of alkyl glyceryl ether and a gum may provide enhanced sweat reduction. For example, in certain embodiments, the deodorant composition may be in the form of a stick and the amount of glycerin may be from about 1 wt. % to about 20 wt. % based on the total weight of the deodorant composition—including all percentages and sub-ranges there-between. In some embodiments, glycerin may be present in an amount ranging from about 2 wt. % to about 19 wt. % based on the total weight of the deodorant composition—including all percentages and sub-ranges there-between. In some embodiments, glycerin may be present in an amount ranging from about 3 wt. % to about 18 wt. % based on the total weight of the deodorant composition—including all percentages and sub-ranges there-between. In some embodiments, glycerin may be present in an amount ranging from about 4 wt. % to about 17 wt. % based on the total weight of the deodorant composition—including all percentages and sub-ranges there-between. In some embodiments, glycerin may be present in an amount ranging from about 5 wt. % to about 16 wt. % based on the total weight of the deodorant composition—including all percentages and sub-ranges there-between. In some embodiments, glycerin may be present in an amount ranging from about 6 wt. % to about 15 wt. % based on the total weight of the deodorant composition—including all percentages and sub-ranges there-between. In some embodiments, glycerin may be present in an amount ranging from about 7 wt. % to about 14 wt. % based on the total weight of the deodorant composition—including all percentages and sub-ranges there-between. In some embodiments, glycerin may be present in an amount ranging from about 8 wt. % to about 13 wt. % based on the total weight of the deodorant composition—including all percentages and sub-ranges there-between. In some embodiments, glycerin may be present in an amount ranging from about 9 wt. % to about 12 wt. % based on the total weight of the deodorant composition—including all percentages and sub-ranges there-between. In some embodiments, glycerin may be present in an amount ranging from about 10 wt. % to about 11 wt. % based on the total weight of the deodorant composition—including all percentages and sub-ranges there-between.

The deodorant composition may comprise hygroscopic agents or humectant agents. Generally, humectants may help bind water in the skin and keep it there for a longer period of time. Humectants may attract and trap water. In some embodiments, the polyols and/or humectants are selected from among glycerin (such as, vegetable refined glycerin), polyhydric alcohols, low molecular weight polyethylene glycols (PEGs) and polyoxyethylenes. Examples of polyhydric alcohols include, but are not limited to, sorbitol (e.g., non-crystal sorbitol), xylitol, propylene glycol, dipropylene glycol, polypropylene glycols (having three or more propylene monomer units), ethylene glycol, diethylene glycol, hexylene glycol, butylene glycol, and combinations thereof. In some embodiments, the polyols and/or humectants are selected from glycerin (e.g., vegetable glycerin), propylene glycol, butylene glycol, polyethylene glycol, 1,2-pentylene glycol and/or a combination thereof.

High molecular weight PEGs may, in some cases, be suitable as polyols and/or humectants, including those having an average molecular weight of about 200,000 [Da] to about 7,000,000 [Da], for example about 500,000 [Da] to about 5,000,000 [Da] or about 1,000,000 [Da] to about 2,500,000 [Da].

The polyols and/or humectants may be chosen from glycerin, propylene glycol, butylene glycol, and 1,3-propanediol, polyglutamic acid, saccharide isomerate, and combinations thereof. In some embodiments, the hygroscopic agent or the humectant agent is vegetable refined glycerin. In some embodiments, the hygroscopic agent or the humectant agent is polyethylene glycol 600. In some embodiments, the hygroscopic agent or the humectant agent is a combination of polyethylene glycol 600 and vegetable glycerin. Without being limited to any particular theory, in some embodiments by reducing the sweat through moisture retention, the antiperspirant/deodorant compositions reduce the formation of the detrimental bacteria responsible for the malodor.

In some embodiments, the deodorant compositions may comprise hygroscopic agents or humectants from about 5 wt. % to about 70 wt. % of the deodorant composition—including all wt. % and sub-ranges there-between. For example, the amount of humectant or hygroscopic agent present in the antiperspirant/deodorant compositions may be from about 5 to about 30 wt. %, about 5 to about 25 wt. %, about 5 to about 22 wt. %, about 5 to about 20 wt. %, about 5 to about 18 wt. %, about 5 to about 16 wt. %; from about 8 to about 30 wt. %, about 8 to about 25 wt. %, about 8 to about 22 wt. %, about 8 to about 20 wt. %, about 8 to about 18 wt. %, about 8 to about 16 wt. %; from about 10 to about 30 wt. %, about 10 to about 25 wt. %, about 10 to about 22 wt. %, about 10 to about 20 wt. %, about 10 to about 18 wt. %, about 10 to about 16 wt. %; from about 12 to about 30 wt. %, about 12 to about 25 wt. %, about 12 to about 22 wt. %, about 12 to about 20 wt. %, about 12 to about 18 wt. %, about 12 to about 16 wt. %; from about 14 to about 30 wt. %, about 14 to about 25 wt. %, about 14 to about 22 wt. %, about 14 to about 20 wt. %, about 14 to about 18 wt. %, about 14 to about 16 wt. %, including ranges and subranges thereof, based on the total weight of the deodorant composition.

In some embodiments the deodorant compositions of the invention comprise hygroscopic agents or humectants in about 10 wt. % to about 20 wt. % of the deodorant composition-including all wt. % and sub-ranges there-between. In some embodiments, the antiperspirant/deodorant compositions of the invention comprise hygroscopic agents or humectants in about 15 wt. % to about 18 wt. % of the antiperspirant/deodorant composition—including all wt. % and sub-ranges there-between. In some embodiments, the antiperspirant/deodorant compositions of the invention comprise hygroscopic agents or humectants in about 15 wt. % of the deodorant composition. In some embodiments, the antiperspirant/deodorant compositions of the invention comprise hygroscopic agents or humectants in about 10 wt. % of the deodorant composition.

The deodorant composition may further comprise a fatty acid salt. The fatty acid salt may be selected from an alkali metal, alkaline earth metal, and amine salts of C₁₄₋₂₂ fatty acids.

In some embodiments, the fatty acid salt may be formed from an alkali metal. The alkali metal may be sodium. In some embodiments, the C₁₄₋₂₂ fatty acid salt may be selected from salts of myristic, palmitic, stearic, behenic, oleic, linoleic, and linolenic acids and mixtures thereof.

In some embodiments, the C₁₄₋₂₂ fatty acid salt may be selected from sodium stearate, potassium stearate, magnesium stearate, sodium oleate, sodium palmitate, sodium behenate, diethylamine stearate, triethylamine stearate, and triethylemine oleate, and mixtures thereof. In some embodiments, the fatty acid salt may be sodium stearate.

In some embodiments, the deodorant composition may be substantially free of salts of hyaluronic acid. In some embodiments, the deodorant composition may be substantially free of a sodium hyaluronate salt.

The C₁₄₋₂₂ fatty acid salt may be present in an amount ranging from about 1 wt. % to about 12 wt. % based on the total weight of the deodorant composition—including all percentages and sub-ranges there-between. The fatty acid salt may be present in the deodorant composition as a thickener (also referred to a gelling agent or structuring agent). The fatty acid salt may be present when the deodorant composition is stick-type deodorant thereby imparting a solid-state to the deodorant composition. In some embodiments, the fatty acid salt may be sodium stearate and may be present in an amount ranging from about 1 wt. % to about 12 wt. % based on the total weight of the deodorant composition—including all percentages and sub-ranges there-between.

In some embodiments, the C₁₄₋₂₂ fatty acid salt may be present in an amount ranging from about 2 wt. % to about 11 wt. % based on the total weight of the deodorant composition-including all percentages and sub-ranges there-between. In some embodiments, the fatty acid salt may be sodium stearate and may be present in an amount ranging from about 2 wt. % to about 11 wt. % based on the total weight of the deodorant composition—including all percentages and sub-ranges there-between.

In some embodiments, the C₁₄₋₂₂ fatty acid salt may be present in an amount ranging from about 3 wt. % to about 10 wt. % based on the total weight of the deodorant composition-including all percentages and sub-ranges there-between. In some embodiments, the fatty acid salt may be sodium stearate and may be present in an amount ranging from about 3 wt. % to about 10 wt. % based on the total weight of the deodorant composition—including all percentages and sub-ranges there-between.

In some embodiments, the C₁₄₋₂₂ fatty acid salt may be present in an amount ranging from about 4 wt. % to about 9 wt. % based on the total weight of the deodorant composition-including all percentages and sub-ranges there-between. In some embodiments, the fatty acid salt may be sodium stearate and may be present in an amount ranging from about 4 wt. % to about 9 wt. % based on the total weight of the deodorant composition—including all percentages and sub-ranges there-between.

In some embodiments, the C₁₄₋₂₂ fatty acid salt may be present in an amount ranging from about 5 wt. % to about 8 wt. % based on the total weight of the deodorant composition-including all percentages and sub-ranges there-between. In some embodiments, the fatty acid salt may be sodium stearate and may be present in an amount ranging from about 5 wt. % to about 8 wt. % based on the total weight of the deodorant composition—including all percentages and sub-ranges there-between.

In some embodiments, the C₁₄₋₂₂ fatty acid salt may be present in an amount ranging from about 6 wt. % to about 7 wt. % based on the total weight of the deodorant composition-including all percentages and sub-ranges there-between. In some embodiments, the fatty acid salt may be sodium stearate and may be present in an amount ranging from about 6 wt. % to about 7 wt. % based on the total weight of the deodorant composition—including all percentages and sub-ranges there-between.

The deodorant composition may comprise an ester of glycerin and a fatty acid selected from C₈₋₁₈ fatty acids. In some embodiments, the C₈₋₁₈ fatty acid may be selected from caprylic, capric, lauric, myristic, palmitic, stearic, oleic, linoleic, and linolenic acids and mixtures thereof. In one embodiment, the fatty acid selected for the glycerin ester may be lauric acid-resulting in glyceryl monolaurate.

The ester of glycerin and the C₈₋₁₈ fatty acids may be present in an amount ranging from about 0.25 wt. % to about 2.25 wt. % based on the total weight of the deodorant composition-including all percentages and sub-ranges there-between. In some embodiments, the glyceryl monolaurate may be present in an amount ranging from about 0.25 wt. % to about 2.25 wt. % based on the total weight of the deodorant composition—including all percentages and sub-ranges there-between.

In some embodiments, the ester of glycerin and the C₈₋₁₈ fatty acids may be present in an amount ranging from about 0.5 wt. % to about 2.0 wt. % based on the total weight of the deodorant composition—including all percentages and sub-ranges there-between. In some embodiments, the glyceryl monolaurate may be present in an amount ranging from about 0.5 wt. % to about 2.0 wt. % based on the total weight of the deodorant composition—including all percentages and sub-ranges there-between.

In some embodiments, the ester of glycerin and the C₈₋₁₈ fatty acids may be present in an amount ranging from about 0.75 wt. % to about 1.75 wt. % based on the total weight of the deodorant composition—including all percentages and sub-ranges there-between. In some embodiments, the glyceryl monolaurate may be present in an amount ranging from about 0.75 wt. % to about 1.75 wt. % based on the total weight of the deodorant composition—including all percentages and sub-ranges there-between.

In some embodiments, the ester of glycerin and the C₈₋₁₈ fatty acids may be present in an amount ranging from about 1.0 wt. % to about 1.5 wt. % based on the total weight of the deodorant composition—including all percentages and sub-ranges there-between. In some embodiments, the glyceryl monolaurate may be present in an amount ranging from about 1.0 wt. % to about 1.5 wt. % based on the total weight of the deodorant composition—including all percentages and sub-ranges there-between.

In some embodiments, the ester of glycerin and the C₈₋₁₈ fatty acids may be present in an amount of about 1.25 wt. % based on the total weight of the deodorant composition. In some embodiments, the glyceryl monolaurate may be present in an amount of about 1.25 wt. % based on the total weight of the deodorant composition.

The deodorant composition may comprise a liquid carrier. The liquid carrier may comprise one or more of a solvent, diluent, a polyol, a humectant, a ester of glycerin, or a combination thereof. The liquid carrier may comprise one or more of water, alcohols (e.g., ethanol, isopropanol, or mixtures thereof), and mixtures thereof. In at least one embodiment, the carrier comprises water.

The amount of liquid carrier (e.g., water) present in the personal care formulation may vary, e.g., from about 5 to about 90 wt. %, based on the total weight of the deodorant composition—including all wt. % and sub-ranges there-between. The liquid carrier content may be selected to produce a stable deodorant composition to make a 100 wt. %, based on the weight of the deodorant composition after all of the materials, including any optional materials, are added to the composition in their desired weight percentages.

In some embodiments, the liquid carrier (e.g., water) may be present in an amount ranging from about 15 wt. % to about 35 wt. % based on the total weight of the deodorant composition-including all percentages and sub-ranges there-between. In some embodiments, water may be present in an amount ranging from about 15 wt. % to about 35 wt. % based on the total weight of the deodorant composition—including all percentages and sub-ranges there-between.

In some embodiments, the liquid carrier may be present in an amount ranging from about 18 wt. % to about 32 wt. % based on the total weight of the deodorant composition—including all percentages and sub-ranges there-between. In some embodiments, water may be present in an amount ranging from about 18 wt. % to about 32 wt. % based on the total weight of the deodorant composition—including all percentages and sub-ranges there-between.

In some embodiments, the liquid carrier may be present in an amount ranging from about 21 wt. % to about 29 wt. % based on the total weight of the deodorant composition—including all percentages and sub-ranges there-between. In some embodiments, water may be present in an amount ranging from about 21 wt. % to about 29 wt. % based on the total weight of the deodorant composition—including all percentages and sub-ranges there-between.

In some embodiments, the liquid carrier may be present in an amount ranging from about 24 wt. % to about 26 wt. % based on the total weight of the deodorant composition—including all percentages and sub-ranges there-between. In some embodiments, water may be present in an amount ranging from about 24 wt. % to about 26 wt. % based on the total weight of the deodorant composition—including all percentages and sub-ranges there-between.

In some embodiments, the liquid carrier may be present in an amount of about 25 wt. % based on the total weight of the deodorant composition. In some embodiments, water may be present in an amount of about 25 wt. % based on the total weight of the deodorant composition.

The deodorant composition may further comprise a thickener. The thickeners may be chosen from polysaccharides; carboxylic acid polymers; crosslinked polyacrylate polymers; polyacrylamide polymers; gums, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some embodiments, the deodorant compositions of the invention comprise two or more thickeners, such as those selected from hydroxypropyl methylcellulose and cross-linked homopolymer of acrylic acid (e.g., Carbopol® Ultrez 30). In some embodiments, the deodorant compositions includes two, three, four, or five thickeners, or any range or subrange thereof. The thickener may be selected from hydroxypropyl methylcellulose, polyethylene glycols and combinations thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

In certain cases, the deodorant composition may be formulated to including thickeners selected from thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and/or starch derivatives (for example, starch oxide, dialdehyde starch, dextrin, British gum, acetyl starch, starch phosphate, carboxymethyl starch, hydroxyethyl starch, hydroxypropyl starch). In some embodiments, the thickeners are selected from among hydroxypropyl methylcellulose, polyethylene glycols (PEGs), polyacrylic acids (sold under the name of Carbopol), cross-linked homopolymer of acrylic acid (sold under the name of Carbopol® Ultrez 30 polymer), and acrylates of C₁₀₋₃₀ alkyl acrylate crosspolymer (sold under the name of Carbopol® Ultrez 20 polymer).

The deodorant compositions of the invention comprise thickeners from about 1 to about 10 wt. % of the total weight of the deodorant composition. For example, the antiperspirant/deodorant composition may include one or more thickener(s) in an amount from about 0.5 wt. % to about 10 wt. %, about 0.5 wt. % to about 8 wt. %, about 0.5 wt. % to about 6 wt. %, about 0.5 wt. % to about 4 wt. %, about 0.5 wt. % to about 2 wt. %, about 0.5 wt. % to about 1 wt. %; from about 1 wt. % to about 10 wt. %, about 1 wt. % to about 8 wt. %, about 1 wt. % to about 6 wt. %, about 1% to about 4 wt. %, about 1 wt. % to about 2 wt. %; from about 2 wt. % to about 10 wt. %, about 2 wt. % to about 8 wt. %, about 2 wt. % to about 6 wt. %, about 2 wt. % to about 4 wt. %; from about 4 wt. % to about 10 wt. %, about 4 wt. % to about 8 wt. %, about 4 wt. % to about 6 wt. %; from about 6 wt. % to about 10 wt. %, about 6 wt. % to about 8 wt. %; from about 8 wt. % to 10 wt. %, or any range or subrange thereof, based on the total weight of the deodorant composition.

The thickener may comprise a gum. The gum may comprise xanthan gum. The gum may be present in an amount ranging from about 0.1 wt. % to about 0.6 wt. % based on the total weight of the deodorant composition—including all percentages and sub-ranges there-between. In some embodiments, the gum may be xanthan gum and be present in an amount ranging from about 0.1 wt. % to about 0.6 wt. % based on the total weight of the deodorant composition—including all percentages and sub-ranges there-between.

In some embodiments, the gum may be present in an amount ranging from about 0.2 wt. % to about 0.4 wt. %, based on the total weight of the deodorant composition—including all percentages and sub-ranges there-between. In some embodiments, the gum may be xanthan gum and be present in an amount ranging from about 0.2 wt. % to about 0.4 wt. %, based on the total weight of the deodorant composition—including all percentages and sub-ranges there-between.

In some embodiments, the gum may be present in an amount ranging from about 0.25 wt. % to about 0.35 wt. %, based on the total weight of the deodorant composition—including all percentages and sub-ranges there-between. In some embodiments, the gum may be xanthan gum and be present in an amount ranging from about 0.25 wt. % to about 0.35 wt. %, based on the total weight of the deodorant composition—including all percentages and sub-ranges there-between.

In some embodiments, the gum may be present in an amount of about 0.3 wt. %, based on the total weight of the deodorant. In some embodiments, the gum may be xanthan gum and be present in an amount of about 0.3 wt. %, based on the total weight of the deodorant composition.

The deodorant composition may further comprise an alkyl glyceryl ether. The alkyl glyceryl ether may comprise one or more of 2-ethylhexylglycerin, isodecyl glyceryl ether, and isostearyl glyceryl ether, and methylheptylglycerin. In some embodiments, the alkyl glyceryl ether is methylheptylglycerin.

The alkyl glyceryl ether may be present in an amount ranging from about 0.5 wt. % to about 2.5 wt. %, based on the total weight of the deodorant composition—including all percentages and sub-ranges there-between. In some embodiments, the alkyl glyceryl ether may be methylheptylglycerin and be present in an amount ranging from about 0.5 wt. % to about 2.5 wt. 9%, based on the total weight of the deodorant composition—including all percentages and sub-ranges there-between.

In some embodiments, the alkyl glyceryl ether may be present in an amount ranging from about 1.0 wt. % to about 2.0 wt. %, based on the total weight of the deodorant composition-including all percentages and sub-ranges there-between. In some embodiments, the alkyl glyceryl ether may be methylheptylglycerin and be present in an amount ranging from about 1.0 wt. % to about 2.0 wt. %, based on the total weight of the deodorant composition—including all percentages and sub-ranges there-between.

In some embodiments, the alkyl glyceryl ether may be present in an amount of about 1.5 wt. %, based on the total weight of the deodorant. In some embodiments, the alkyl glyceryl ether may be methylheptylglycerin and be present in an amount of about 1.5 wt. %, based on the total weight of the deodorant composition—including all percentages and sub-ranges there-between.

The alkyl glyceryl ether and the gum may be present in a weight ratio ranging from about 1:1 to about 7:1—including all ratios and sub-ranges there-between. In some embodiments, the alkyl glyceryl ether and the gum may be present in a weight ratio ranging from about 3:1 to about 6:1—including all ratios and sub-ranges there-between. In some embodiments, the alkyl glyceryl ether and the gum may be present in a weight ratio of about 5:1.

In certain preferred embodiments, the deodorant composition comprises the combination of an alkyl glyceryl ether, glycerin, and a gum in certain amounts and ratios thereof. For instance, it has been surprisingly discovered that certain embodiments of the deodorant composition comprising particular thickeners and alkyl glyceryl ethers may provide an unexpected improvement in malodor reduction after application to a user's skin. Additionally and/or alternatively, it was unexpectedly discovered that certain amounts of glycerin in combination with certain amounts of alkyl glyceryl ether and a gum may provide enhanced sweat reduction. For example, it has been discovered that a stick-type deodorant (as discussed in greater detail herein) comprising glycerin in certain amounts and in combination with particular alkyl glyceryl ethers exhibit an unexpected improvement in sweat reduction and/or malodor reduction after application to a user's skin. In at least one preferred embodiment, deodorant compositions of the present invention comprising glycerin, xanthan gum, and methylheptylglycerin in certain amounts and ratios thereof exhibit an unexpected improvement in sweat reduction malodor reduction after application to a user's skin. For instance, it has been discovered that a stick-type deodorant comprising the blend of xanthan gum and that methylheptylglycerin exhibits an unexpected improvement in malodor reduction after application to a user's skin. In some embodiments, xanthan gum and methylheptylglycerin are blended together prior to combining with other ingredients of the deodorant composition.

The deodorant composition may further comprise an additive. In a non-limiting example, the additive may be selected from one or more of a soothing agent, a fragrance, antioxidant, and a colorant. Non-limiting examples of fragrances include lemongrass oil, apricot fragrance, fir needle oil, lavandin oil and lavender oil. The deodorant compositions of the invention comprise fragrances from about 0.01 to about 10 wt. %, relative to the total weight of the deodorant composition. The deodorant compositions of the invention comprise fragrances from about 0.1 to about 2 wt. %, based on the total weight of the deodorant composition. The deodorant compositions of the invention comprise fragrances from about 0.5 to 1.1 wt. %, based on the total weight of the antiperspirant/deodorant composition.

Non-limiting examples of soothing agents include aloe vera, herbal extracts, and witch hazel water. Non-limiting examples of antioxidants are, for example, one or more of tocopherol and its derivatives, butyl hydroxyanisole (BHA), butyl hydroxytoluene (BHT), erythorbic acid, propyl gallate, sodium erythorbate, tertiary butyl hydroquinone (TBHQ), rosemary extract and, more preferably, ascorbic acid and salts thereof.

The deodorant composition may comprise one or more alpha hydroxy acids, beta hydroxy acids, polyhydroxy acids or combinations thereof.

In a non-limiting embodiment, the deodorant composition may include one or more alpha hydroxy acid(s), such as those selected from C₃ to C₇ alpha-hydroxy acid or C₄ to C₆ alpha-hydroxy acid. In some embodiments, the personal care formulation includes a salt of an alpha-hydroxy acid. The salt of the alpha-hydroxy acid is preferably a sodium salt or a potassium salt. In at least one embodiment, the salt is a sodium salt (i.e., the cation associated with the salt is a sodium).

Non-limiting examples of alpha hydroxy acids include but are not limited to, mandelic acid, glycolic acid, citric acid, lactic acid, malic acid, tartaric acid, phytic acid, hydroxycaprylic acid, hydroxycapric acid, glutaric acid, gluconic acid, or a combination of two or more thereof. In some embodiments, the alpha hydroxy acids are selected from among mandelic acid, glycolic acid, citric acid, lactic acid, malic acid, tartaric acid, phytic acid, hydroxycaprylic acid, hydroxycapric acid and combinations thereof. In further embodiments, the alpha hydroxy acids are citric acid, mandelic acid, glycolic acid, lactic acid or a combination of two or more thereof. In certain instances, the alpha-hydroxy acid is selected from malic acid, tartaric acid, alpha-hydroxy glutaric acid, gluconic acid, a salt thereof, and a combination of two or more thereof. Yet in further instances, the alpha-hydroxy acid or salt thereof is selected from lactic acid, malic acid, and sodium-D-gluconate. In some embodiments, the alpha hydroxy acid is lactic acid. In some embodiments the alpha hydroxy acid is lactic acid. The antiperspirant/deodorant compositions of the invention comprise alpha hydroxy acids from about 0.05 to about 2 wt. %, based on the total weight of the antiperspirant/deodorant composition.

The amount of alpha hydroxyl acid in the deodorant compositions may be from about 0.05 to about 2 wt. %, about 0.05 to about 1.8 wt. %, about 0.05 to about 1.6 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 2 wt. %, about 0.1 to about 1.8 wt. %, about 0.1 to about 1.6 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %; from about 0.3 to about 2 wt. %, about 0.3 to about 1.8 wt. %, about 0.3 to about 1.6 wt. %, about 0.3 to about 1.4 wt. %, about 0.3 to about 1.2 wt. %, about 0.3 to about 1 wt. %, about 0.3 to about 0.8 wt. %; from about 0.6 to about 2 wt. %, about 0.6 to about 1.8 wt. %, about 0.6 to about 1.6 wt. %, about 0.6 to about 1.4 wt. %, about 0.6 to about 1.2 wt. %, about 0.6 to about 1 wt. %; from about 0.9 to about 2 wt. %, about 0.9 to about 1.8 wt. %, about 0.9 to about 1.6 wt. %, about 0.9 to about 1.4 wt. %; from about 1.2 to about 2 wt. %, about 1.2 to about 1.8 wt. %, about 1.2 to about 1.6 wt. %; from about 1.5 to about 2 wt. %, about 1.5 to about 1.8 wt. %, or any range or subrange thereof, based on the total weight of the antiperspirant/deodorant composition. The deodorant compositions of the invention comprise alpha hydroxy acids from about 0.1 to about 0.15 wt. %, from about 0.15 to about 1 wt. %, or from about 1 to about 2 wt. %, based on the total weight of the deodorant composition. The deodorant compositions of the invention comprise alpha hydroxy acids in about 0.15 wt. %, based on the total weight of the deodorant composition.

Non-limiting examples of beta hydroxy acids include salicylic acid, propionic acid, beta-hydroxybutyric acid, beta-hydroxy beta-methylbutyric acid, carnitine, and combinations of two or more thereof. The beta hydroxy acids may in some cases be selected from salicylic acid, esters of salicylic acid, sodium salicylate, beta hydroxybutanoic acid, tropic acid, trethocanic acid, beta hydroxyl acids obtained from white willow bark extract and/or wintergreen leaves, and combinations of two or more thereof. In some embodiments, the beta hydroxy acid(s) comprises salicylic acid, sodium salicylate, beta hydroxyl acids of willow bark extract or combinations thereof. In some embodiments, the beta hydroxy acid is salicylic acid.

Non-limiting examples of polyhydroxy acids may include, but are not limited to, gluconolactone, gluconic acid, galactose, lactobionic acid, or combinations thereof. In some embodiments, the polyhydroxy acids are gluconolactone, lactobionic acid or combinations thereof. For example, the polyhydroxy acid may be glucanodeltalactone.

The amount of polyhydroxy acid in the deodorant compositions may be from about 0.05 to about 2 wt. %, about 0.05 to about 1.8 wt. %, about 0.05 to about 1.6 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 2 wt. %, about 0.1 to about 1.8 wt. %, about 0.1 to about 1.6 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %; from about 0.3 to about 2 wt. %, about 0.3 to about 1.8 wt. %, about 0.3 to about 1.6 wt. %, about 0.3 to about 1.4 wt. %, about 0.3 to about 1.2 wt. %, about 0.3 to about 1 wt. %, about 0.3 to about 0.8 wt. %; from about 0.6 to about 2 wt. %, about 0.6 to about 1.8 wt. %, about 0.6 to about 1.6 wt. %, about 0.6 to about 1.4 wt. %, about 0.6 to about 1.2 wt. %, about 0.6 to about 1 wt. %; from about 0.9 to about 2 wt. %, about 0.9 to about 1.8 wt. %, about 0.9 to about 1.6 wt. %, about 0.9 to about 1.4 wt. %; from about 1.2 to about 2 wt. %, about 1.2 to about 1.8 wt. %, about 1.2 to about 1.6 wt. %; from about 1.5 to about 2 wt. %, about 1.5 to about 1.8 wt. %, or any range or subrange thereof, based on the total weight of the deodorant composition.

In some embodiments, the deodorant compositions of the invention comprise anti-foaming agents or preservatives. The deodorant compositions may comprise preservatives selected from among caprylyl glycol, phenoxyethanol, butylated hydroxytoluene (BHT), ethylenediaminetetraacetic acid (EDTA), ethylhexylglycerin, citric acid, benzoic acid, and combinations thereof. In some embodiments the anti-foaming agent or preservative is caprylyl glycol. Caprylyl glycol may also be referred to as 1,2-octanediol.

The deodorant compositions of the invention comprise anti-foaming agents or preservatives from about 0.1 wt. % to about 10 wt. %, based on the total weight of the deodorant composition—including all amounts and sub-ranges there-between.

In some embodiments, the antiperspirant/deodorant compositions comprise optional ingredients such as fragrances, surfactants, plant based oils, antioxidants, antibacterial agents, deodorizing agents, skin soothing agents, vitamins, pH adjusters, polymers, or combinations thereof.

In some embodiments, the deodorant compositions of the invention comprise surfactants. In some embodiments, the deodorant compositions of the invention comprise surfactants selected from among nonionic surfactants, anionic surfactants, cationic surfactants and amphoteric/zwitterionic surfactants. Examples of anionic surfactants include, but are not limited to, ammonium lauryl sulfate, ammonium lauryl ether sulfate, sodium lauryl sulfate, sodium lauryl ether sulfate, or a combination of two more thereof. For example, the anionic surfactant may be selected from ammonium lauryl sulfate, ammonium lauryl ether sulfate, and a combination of two or more thereof.

The amount of anionic surfactants in the deodorant composition may be about 0.5 to about 10 wt. %, based on the total weight of the personal care composition. In some instances, the deodorant composition include anionic surfactants in an amount from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 4 to about 10 wt. %, about 4 to about 8 wt. %, about 4 to about 6 wt. %; from about 6 to about 10 wt. %, about 6 to about 8 wt. %; from about 8 to 10 wt. %, or any range or subrange thereof, based on the total weight of the deodorant composition.

The amphoteric surfactants in the antiperspirant/deodorant compositions may be selected from a betaine, an alkyl sultaine, an alkyl amphoacetate, an alkyl amphoproprionate, a salt thereof, and a combination of two or more thereof. Preferably, the amphoteric surfactant comprises a betaine surfactant. Non-limiting examples of betaine surfactants include cocamidopropyl betaine, cocodimethylcarboxymethylbetaine, lauryldimethylcarboxymethylbetaine, lauryldimethylcarboxyethylbetaine, cetyldimethyl-carboxymethylbetaine, lauryl-bis-(2-hydroxyethyl)-carboxymethylbetaine, olcyl-dimethylgammacarboxypropylbetaine, and lauryl-bis-(2-hydroxypropyl)-carboxyethyl-betaine, or a combination of two or more thereof.

The amount of amphoteric surfactants in the deodorant composition may be about 0.5 to about 10 wt. %, based on the total weight of the personal care composition. In some instances, the antiperspirant/deodorant composition include amphoteric surfactants in an amount from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 4 to about 10 wt. %, about 4 to about 8 wt. %, about 4 to about 6 wt. %; from about 6 to about 10 wt. %, about 6 to about 8 wt. %; from about 8 to 10 wt. %, or any range or subrange thereof, based on the total weight of the deodorant composition.

In some embodiments, the surfactants are nonionic surfactants. In some embodiments, the nonionic surfactants include, but are not limited to, secondary alcohol ethoxylates, polyoxyethylene stearyl ethers, polyoxyethylene ethers, a mixture of high molecular mass saturated fatty alcohols, mainly cetyl alcohol and stearyl alcohol. The antiperspirant/deodorant compositions may include a nonionic surfactant blend including two or more fatty alcohol ethoxylates, each having a polyethylene oxide chain length of at least 2. Preferred fatty alcohol ethoxylates generally have a fatty alcohol chain length of C₁₂ to C₂₄, a degree of unsaturation of 0-2, and a polyethylene oxide chain length of 2 to 150 ethylene oxide units. For example, the fatty alcohol ethoxylates may have the general formula: CH₃—(CH₂)ₓ—CH₂—O—(CH₂—CH₂—O—)_{y}H—, where X=10-20, Y=2-150. In some cases, the blend of fatty alcohol ethoxylates includes at least one fatty alcohol ethoxylate having a long polyethylene oxide chain length and at least one fatty alcohol ethoxylate having a short polyethylene oxide chain length. Suitable long chain length fatty alcohol ethoxylates have a polyethylene oxide chain length greater than 20, preferably 21 to 150, more preferably 21 to 100. For example, the fatty alcohol ethoxylate may be a polyoxyethylene stearyl ethers having a structuring according to the chemical formula: CH₃—(CH₂)₁₆—CH₂—(O—CH₂—CH₂)ₙ—OH with average n being 2 to 20, respectively.

Preferred long chain fatty alcohol ethoxylates include Steareth-100 (100 indicates the polyethylene oxide chain length) and Steareth 21. Other long chain fatty alcohol ethoxylates may be used, e.g., Ceteth-100, Oleth-100 Myreth-100, and Beheneth-100. These surfactants have a preferred HLB range from 15 to 8. Suitable short chain length fatty alcohol ethoxylates have a polyethylene oxide chain length of less than or equal to 20, preferably 2 to 20. Suitable shorter chain length fatty alcohol ethoxylates include, for example, Steareth-2, Steareth-10, Ceteth-20, Steareth-20, Myreth-20, Oleth-20 and Beheneth-20. These surfactants have a preferred HLB range from 4 to 16.

The non-ionic surfactants of the deodorant composition may be selected from polyoxyethylene ethers, such as polyoxyethylene stearyl ethers compounds. The polyoxyethylene ethers may comprise a mixture of high molecular mass saturated fatty alcohols, such as cetyl alcohol and stearyl alcohol, and ethylene oxide. The polyoxyethylene ethers may be selected from Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, and a combination of two or more thereof. Examples of polyoxyethylene stearyl ethers compounds include Steareth-2, Steareth-4, Steareth-6, Steareth-7, Steareth-21, Steareth-10, Steareth-11, Steareth-13, Steareth-15, Steareth-20, or combinations of two or more thereof. Certain secondary alcohol ethoxylates are sold under the trade names of Tergitol®, available from Dow™.

The amount of nonionic surfactants (e.g., polyoxyethylene ethers and/or polyoxyethylene stearyl ethers) in the antiperspirant/deodorant composition may be about 0.5 to about 10 wt. %, based on the total weight of the deodorant composition. In some instances, the deodorant composition include nonionic surfactants (e.g., polyoxyethylene ethers and/or polyoxyethylene stearyl ethers) in an amount from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 4 to about 10 wt. %, about 4 to about 8 wt. %, about 4 to about 6 wt. %; from about 6 to about 10 wt. %, about 6 to about 8 wt. %; from about 8 to 10 wt. %, or any range or subrange thereof, based on the total weight of the deodorant composition. In some cases, the deodorant compositions may comprise surfactants from about 0.1 to about 5 wt. %, based on the total weight of the deodorant composition. The deodorant compositions of the invention comprise surfactants from about 1 to about 3 wt. %, based on the total weight of the deodorant composition. The deodorant compositions of the invention comprise surfactants in about 2 wt. %, based on the total weight of the antiperspirant/deodorant composition.

The deodorant compositions of the invention may comprise Tergitol® in about 2 to about 5 wt. %, based the total weight of the deodorant composition. The deodorant compositions of the invention comprise Tergitol® in about 2 wt. %, based on the total weight of the deodorant composition. The antiperspirant/deodorant compositions of the invention comprise Tergitol® in about 5 wt. %, based on the total weight of the deodorant composition.

In some embodiments, the deodorant compositions of the invention comprise antioxidants. The deodorant compositions of the invention comprise antioxidants from about 1 to about 5 wt. %, based on the total weight of the deodorant composition. For instance, the amount of antioxidant present in the deodorant composition may be from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %; from about 4 to about 5 wt. %, or any range or subrange thereof, based on the total weight of the deodorant composition. In some embodiments, the deodorant compositions of the invention comprise antioxidants selected from among witch hazel, butylated hydroxytoluene, pentaerythrityl tetra-di-t-butyl hydroxyhydrocinnamate or combinations thereof. Pentaerythrityl tetra-di-t-butyl hydroxyhydrocinnamate is commercially available under the name of Tinogard TT™ by Ciba/BASF. In some embodiments, the deodorant compositions of the invention comprise witch hazel in about 2 wt. %, relative to the total weight of the composition.

The pH of the antiperspirant/deodorant composition can be in the range of 4 to 11, or 7.5 to 10.5, or 8 to 10, or 8.5 to 9.5, or 9 to 9.5 or 9 to 10.

The deodorant composition may be in a solid state at room temperature (i.e., about (20° C. to about 22° C.). The term solid state may refer to the deodorant composition having a self-supporting structure that can maintain a predetermined shape without the assistance of a container. In the solid-state, the deodorant composition may be used in a stick-type deodorant (as compared to a liquid-state roll-on type deodorant).

The stick-type deodorant may be glide-applied to the skin of a user (as compared to roll apply). During glide application, portions of the deodorant composition are deposited onto the user's skin and remain on the user skin for a period of time to impart malodor protection to that portion of the skin. During glide application, the deodorant composition may be in the solid-state at room temperature.

The deodorant composition of the present invention may be manufactured by mixing together the aforementioned components to create a blend. The blend may be heated to an elevated temperature that is above room temperature. In a non-limiting embodiment, the elevated temperature may range from about 75° F. to about 185° F.—including all temperatures and sub-ranges there-between. In a non-limiting embodiment, the elevated temperature may be such that the blend is in a liquid-state or a semi-liquid state to help ensure even distribution of each component throughout the blend. The term “liquid state” or “semi-solid” state may refer to a composition that is able to flow and not retain a self-supporting structure and maintain a predetermined shape without the assistance of a container.

A predetermined amount of the blend may then be deposited into an empty deodorant container, whereby the heated blend may then be allowed to cool to at least room temperature, thereby solidifying the blend into the solid state.

In a non-limiting embodiment, the blend may be formed by mixing together the liquid carrier; the polyols; the alkyl glyceryl ether; the xanthan gum; an ester of glycerin and a C₈₋₁₈ fatty acid; and the alkali salt of a C₁₄₋₂₂ fatty acid. The blend may further comprise mixing in the additive.

### EXAMPLES

The following experiments tested the impact on deodorant efficacy based on the presence of methylheptylglycerin (“MHG”) and xanthan gum (“XG”). The formulation of each example is set forth below in Table 1—each numerical value represents a weight percentage (wt. %).

### Example 1

An experiment was performed to test the impact of malodor reduction over time using the deodorant formulation of the present invention-formulations are set forth below in Table 1.

TABLE 1

| | Comp. | Comp. | | |
|---|---|---|---|---|
| | Ex. 1 | Ex. 2 | Ex. 1 | Ex. 2 |
| Ingredients | (wt. %) | (wt. %) | (wt. %) | (wt. %) |
| Water | 25.35 | 24.5 | 24.5 | 24.5 |
| Butylene Glycol | — | — | — | 42.2 |
| Propylene Glycol | 56.28 | 56.73 | 56.43 | 14.23 |
| Sodium Stearate | 6.03 | 6.0 | 6.0 | 6.0 |
| Glyceryl Monolaurate | 1.26 | 1.25 | 1.25 | 1.25 |
| Glycerin | 10.05 | 10.0 | 10.0 | 10.0 |
| Sodium Hyaluronate | 1.01 | — | — | — |
| Solution 1% | | | | |
| MHG | — | 1.5 | 1.5 | 1.5 |
| XG | — | — | 0.3 | 0.3 |
| Aloe Vera | 0.02 | 0.02 | 0.02 | 0.2 |

Each of Comparative Example 1 (Comp. Ex. 1), Comparative Example 2 (Comp. Ex. 2), Example 1 (Ex. 1), and Example 2 (Ex. 2) were then subject to a malodor test to measure the deodorant efficacy of each formulation. The clinical malodor test was performed on male participants between 18 and 55 years old who were subject to a “sniff evaluation” of malodor of the underarm area by trained judges using a 0 to 10 odor intensity scale (0-no malodor, 5=moderate malodor, 10-extremely strong malodor). Subjects with a malodor score of 5 or greater at baseline (when using no deodorant formulation) were qualified to participate in the test. After a wash of the underarm area and the application of the tested products, the malodor was judged after a period of 12 hours, 24 hours, and 48 hours.

The results of the test are set forth below in Table 2.

TABLE 2

| | Baseline | 12 | 24 | 48 |
|---|---|---|---|---|
| | Malodor Score | Hours | Hours | Hours |
| Comp. Ex. 1 | 6.31 | 2.17 | 3.17 | 5.23 |
| Comp. Ex. 2 | 6.31 | 1.49 | 2.48 | 4.54 |
| Ex. 1 | 6.31 | 0.84 | 1.85 | 3.87 |
| Ex. 2 | 6.58 | 1.12 | 1.92 | 3.14 |

As demonstrated by the date described in Table 2 (above), it was surprisingly observed that the two exemplary compositions of the present invention (Ex. 1 and Ex. 2) exhibited an unexpected improvement in malodor reduction for each recorded time period, with an extremely significant reduction in malodor over the span of 48 hours. Specifically, not only did the exemplary compositions of the present invention (Ex. 1 and Ex. 2) exhibit a surprisingly large drop in malodor protection in the first 12 hours, but the deodorant compositions continued to outperform the comparative compositions over the following 36 hours period (i.e., up to 48 hours).

### Example 2

Following a 21-day conditioning period requiring the discontinuation of all axillary antiperspirants, male subjects (½ with shaved axilla, ½ unshaved), age 18-65, reported to the test facility for consent and screening. Seventy-two (72) male subjects who met the study criteria were enrolled and participated in a baseline sweat collection during daytime. Sixty-four (64) male subjects with sweat collections of at least 100 mg of sweat per axilla were permitted to continue and were assigned a final number in order of the highest sweater to the lowest sweater.

Subjects that were assigned a final number participated in a supervised wash and test article application. These subjects returned from Days 2 to Day 8 for axillary evaluation, supervised wash and test article application. On Day 8 subjects also participated in a sweat collection, 1 hour after the 8th application. On Day 9 subjects reported for axillary evaluation and sweat collection (24 hours after the 8th application). On Day 10 subjects reported for axillary evaluation and sweat collection (48 hours after the 8th application). Sixty-one (61) subjects completed the study. Two (2) subjects dropped out from the study.

Sweating of the subjects was induced by having the subjects sit outside under ambient conditions for 4 hours sweat collection during morning time. This method of sweat collection is permitted under the guidelines for Effectiveness Testing of OTC Antiperspirant Drug Products, June 2003.1

Data were analyzed following the FDA guidelines for Effectiveness Testing of OTC Antiperspirant Drug Products. The Ratio Method was used to determine % sweat reduction for an exemplary composition of the present invention that contained 10% Glycerin (Ex. 3) relative to the untreated underarm. The formula for Ex. 3 is described in Table 3 (below).

TABLE 3

| Ingredient | Ex. 3 (Wt. %) |
|---|---|
| Propylene Glycol | 58.8 |
| Water | 26.9 |
| Glycerin | 10 |
| Sodium Stearate | 6 |
| Glyceryl Monolaurate | 1.3 |
| Ascorbic Acid | 0.05 |
| Aloe Vera Gel - Spray Dried | 0.03 |

The Wilcoxon Signed Rank test was used to determine significance level for 20% sweat reduction (Z<0.8) and for 30% sweat reduction (Z<0.7) at p<0.05 significance level. It was also used to determine the general efficacy requirement (Z<1.0) at 48 hours only.

Based on the results of the Wilcoxon Signed Rank Test, the exemplary composition of the present invention (Ex. 3) was significantly effective in reducing perspiration at 1 hour after Application No. 8 when tested against both the Z<0.8 hypothesis (20% sweat reduction) and the Z<0.7 hypothesis (30% sweat reduction). Further, Ex. 3 was significantly effective in reducing perspiration at 24 hours only for Z<0.8 hypothesis (20% sweat reduction). The mean percent sweat reductions, the 95% confidence intervals, the significance levels derived from the distribution-free signed rank test, are presented in Table 4 (below).

TABLE 4

| | % Sweat | | Significance Level | | |
|---|---|---|---|---|---|
| | Reduction vs. | 95% | | | Hₐ: Z < 1.0 |
| | Untreated | Confidence | Hₐ: Z < 0.8 | Hₐ: Z < 0.7 | General |
| Evaluation | Axilla | Interval | 20% reduction | 30% reduction | Efficacy |
| 1 Hr. after | 49.17% | 44.03% to 54.30% | <0.05* | <0.05* | <0.05* |
| Application# 8 | | | | | |
| 24 Hr. after | 30.97% | 25.68% to 36.25% | <0.05* | >0.05 | <0.05* |
| Application# 8 | | | | | |
| 48 Hr. after | 14.20% | 7.16% to 21.23% | >0.05 | >0.05 | <0.05* |
| Application# 8 | | | | | |

*Significantly effective at p < 0.05

As illustrated by the data described in Table 3 (above), an exemplary composition provided sweat reductions of 49.16% at 1 hour, 30.97% at 24 hours and 14.20% at 48 hours.

### Example 3

Following a 21-day conditioning period requiring the discontinuation of all axillary antiperspirants, subjects (half male, half female), age 18-65, reported to the test facility for consent and screening. Sixty (60) subjects who met the study criteria were enrolled. Subjects were assigned to the randomization after satisfying the baseline acceptance criteria.

At Day 1 subjects participated in a supervised wash with a non-antimicrobial soap and an exemplary composition of the present invention (Ex. 4) application. The formula for Ex. 4 is described in Table 5 (below).

TABLE 5

| Ingredients | Ex. 4 (wt. %) |
|---|---|
| Propylene Glycol | 56.3 |
| Water | 25.4 |
| Glycerin | 10.1 |
| Sodium Stearate | 6 |
| Glyceryl Monolaurate | 1.3 |
| Sodium Hyaluronate Solution | 1 |
| Aloe Vera Gel - Spray Dried | 0.03 |

The subjects returned from Days 2 to Day 8 for axillary evaluation, supervised wash and Ex. 4 application. On Day 8 subjects also participated in a sweat collection, 1 hour after the 8^{th} application. On Day 9 subjects reported for axillary evaluation and sweat collection (24 hours after the 8^{th} application). Fifty-one (51) subjects with 23 male and 28 female completed the study. Nine (9) subjects withdrew or were withdrawn from the study. Sweating of the subjects was induced by sitting the subject in a room maintained at 100±2 F and 30%-40% relative humidity (RH).

Data were analyzed following the FDA guidelines for Effectiveness Testing of OTC Antiperspirant Drug Products. The Ratio Method was used to determine % sweat reduction for an exemplary composition of the present invention (Ex. 4) relative to the untreated underarm. The Wilcoxon Signed Rank test was used to determine significance level for 20% sweat reduction (Z<0.8) at p<0.05 significance level. It was also used to determine the general efficacy requirement (Z<1.0).

Based on the results of the Wilcoxon Signed Rank Test, an exemplary composition of the present invention (Ex. 4) was significantly more effective in reducing perspiration at 1 hour after Application No. 8 when tested against both the Z<0.8 hypothesis (20% sweat reduction). The exemplary composition of the present invention (Ex. 4) was significantly more effective in reducing perspiration at 24 hours against the Z<1.0 hypothesis (general efficacy). The mean percent sweat reductions, the 95% confidence intervals, the significance levels derived from the distribution-free signed rank test, are presented in Table 6 (below).

TABLE 6

| | % Sweat | | Significance Level | |
|---|---|---|---|---|
| | Reduction vs. | 95% Con- | Hₐ: Z < 0.8 | Hₐ: Z < 1.0 |
| | Untreated | fidence | 20% | General |
| Evaluation | Axilla | Interval | reduction | Efficacy |
| 1 Hr. after | 22.05% | 13.15% to | <0.05* | <0.05* |
| Application# 8 | | 30.95% | | |
| 24 Hr. after | 9.89% | 3.23% to | >0.05 | <0.05* |
| Application# 8 | | 16.46% | | |

*Significantly effective at p < 0.05

Specifically, an exemplary composition of the present invention demonstrated sweat reductions of 22.05% at 1 hour and 9.89% at 24 hours.

### Example 4

A 3-week at consumer at home evaluation was conducted to assess a non-limiting, example deodorant composition (Ex. 5) in accordance with an aspect of the invention. The formulation of Example Deodorant Composition 5 is shown in Table 7. Specifically, 320 volunteers of ages between 18 to 65 were instructed to apply the deodorant composition to their arm pits as they normally would use a deodorant and/or antiperspirant composition over the course of 3-week period.

TABLE 7

| | Ex. 5 (Wt. %) |
|---|---|
| Water | 22-27 |
| Butylene Glycol | 13-16 |
| Propylene Glycol | 40-45 |
| Sodium Stearate | 5-7.4 |
| Glyceryl Monolaurate | 1-1.6 |
| Glycerin | 8-12 |
| Methylheptylglycerin | 1.25-2 |
| Xanthan Gum | 0.1-0.5 |
| Aloe Vera | 0.01-0.1 |
| Fragrance | 1.1 |

After the 3-week period, each volunteer completed a questionnaire, which was professionally evaluated to determine the outcome of the consumer at home evaluation. Specifically, 88% of the volunteers indicated that Example Composition 5 provided protection against wetness, while 11% of volunteers indicated that Example Composition 5 did not provide protection against wetness and 1% of volunteers were unsure if Example Composition 5 provided protection against wetness.

### Example 5

A monograph study was conducted to assess a non-limiting, example deodorant composition (Ex. 6) in accordance with an aspect of the invention. The formulation of Example Deodorant Composition 6, which is shown in Table 8, was the same as Example Deodorant Composition 5, except that Example Deodorant Composition 6 was fragrance free.

TABLE 8

| | Ex. 6 (Wt. %) |
|---|---|
| Water | 22-27 |
| Butylene Glycol | 13-16 |
| Propylene Glycol | 40-45 |
| Sodium Stearate | 5-7.4 |
| Glyceryl Monolaurate | 1-1.6 |
| Glycerin | 8-12 |
| Methylheptylglycerin | 1.25-2 |
| Xanthan Gum | 0.1-0.5 |
| Aloe Vera | 0.01-0.1 |

Specifically, a dose of 0.45-0.55 g/axilla was applied using fifty-four (54) volunteers. At Day 1 the volunteers participated in a supervised wash with a non-antimicrobial soap and an application of Example Deodorant Composition 6. The volunteers returned from Days 2 to Day 8 for axillary evaluation, supervised wash, application of Example Deodorant Composition 6.

On Day 8, the volunteers participated in a sweat collection, 1 hour after the 8^{th} application. On Day 9, the volunteers reported for axillary evaluation and sweat collection (24 hours after the 8th application). Sweating of the subjects was induced by sitting the subject in a room maintained at 100±2 F and 30%-40% relative humidity (RH).

Data was analyzed following the FDA guidelines for Effectiveness Testing of OTC Antiperspirant Drug Products. The Ratio Method was used to determine % sweat reduction for Example Deodorant Composition 6 relative to the untreated underarm. The Wilcoxon Signed Rank test was used to determine significance level for 20% sweat reduction (Z<0.8) at p<0.05 significance level, for 30% sweat reduction (Z<0.7) at p<0.05 significance level, and for the general efficacy requirement (Z<1.0) at p<0.05 significance level. A summary of the results in shown in Table 9.

TABLE 9

| | Percent | | Significance Level | | |
|---|---|---|---|---|---|
| | Sweat | | Z < 0.8 (20% | Z < 0.7 (30% | Z < 1 |
| | Reduction vs. | Confidence | Sweat | Sweat | (General |
| Evaluation | Untreated | Level | Reduction) | Reduction) | Efficacy) |
| 1 hour after | 31.5% | −1.57%-64.57% | <0.05 | >0.05 | <0.05* |
| application | | | | | |
| no. 8 | | | | | |
| 24 hours after | 11.96% | 3.12%-20.8% | >0.05 | >0.05 | <0.05* |
| application | | | | | |
| no. 8 | | | | | |

*Significantly effective at p < 0.05

While the invention has been described with respect to specific examples including presently preferred modes of carrying out the invention, those skilled in the art will appreciate that there are numerous variations and permutations of the above-described compositions, products, and/or systems. It is to be understood that other embodiments may be utilized and structural and functional modifications may be made without departing from the scope of the present invention. Thus, the spirit and scope of the invention should be construed broadly as set forth in the appended claims.

What is claimed is:

1. A personal care composition comprising:
an alkyl glyceryl ether;
xanthan gum;
an ester of glycerin and a C₈₋₁₈ fatty acid;
a polyol comprising glycerin, and
an alkali salt of a C₁₄₋₂₂ fatty acid.

2. The personal care composition according to claim 1, wherein the alkyl glyceryl ether is present in an amount ranging from about 0.5 wt. % to about 2.5 wt. %, based on the total weight of the personal care composition.

3. The personal care composition according to claim 1, wherein the xanthan gum is present in an amount ranging from about 0.1 wt. % to about 0.6 wt. %, based on the total weight of the personal care composition.

4. The personal care composition according to claim 1, wherein the ester of glycerin and a C₈₋₁₈ fatty acid is present in an amount ranging from about 0.25 wt. % to about 2.25 wt. %, based on the total weight of the personal care composition.

5. The personal care composition according to claim 4, wherein the polyol further comprises a diol selected from propylene glycol, butylene glycol, or a combination thereof, wherein the diol is present in an amount ranging from about 40 wt. % to about 66 wt. %, based on the total weight of the personal care composition.

6. The personal care composition according to claim 1, wherein the alkali salt of the C₁₄₋₂₂ fatty acid is present in an amount ranging from about 1 wt. % to about 12 wt. %, based on the total weight of the personal care composition.

7. The personal care composition according to claim 1 further comprising a liquid carrier comprising water, wherein the liquid carrier is present in an amount ranging from about 15 wt. % to about 35 wt. %, based on the total weight of the personal care composition.

8. The personal care composition according to claim 1, wherein the personal care composition is a deodorant composition, the deodorant composition being in a solid state at room temperature.

9. A personal care composition comprising:
methylheptylglycerin;
xanthan gum;
glyceryl monolaurate;
a polyol comprising a diol and a triol; and
sodium stearate.

10. The personal care composition according to claim 9, wherein the methylheptylglycerin is present in an amount ranging from about 0.5 wt. % to about 2.5 wt. %, based on the total weight of the personal care composition.

11. The personal care composition according to claim 9, wherein the xanthan gum is present in an amount ranging from about 0.1 wt. % to about 0.6 wt. %, based on the total weight of the personal care composition.

12. The personal care composition according to claim 9, wherein the glyceryl monolaurate is present in an amount ranging from about 0.25 wt. % to about 2.25 wt. %, based on the total weight of the personal care composition.

13. The personal care composition according to claim 9, wherein the sodium stearate is present in an amount ranging from about 1 wt. % to about 12 wt. %, based on the total weight of the personal care composition.

14. A personal care composition comprising:
a polyol comprising glycerin;
methylheptylglycerin in an amount ranging from about 0.5 wt. % to about 2.5 wt. %, based on the total weight of the deodorant composition;
xanthan gum in an amount ranging from about 0.1 wt. % to about 0.6 wt. %, based on the total weight of the deodorant composition;
sodium stearate in an amount ranging from about 1 wt. % to about 12 wt. %, based on the total weight of the deodorant composition; and
glyceryl monolaurate in an amount ranging from about 0.25 wt. % to about 2.25 wt. %, based on the total weight of the deodorant composition,
wherein the personal care composition is in the form of a deodorant composition.

15. The personal care composition according to claim 14, wherein the personal care composition is in the solid state and is in the form of a predetermined shape.

16. The personal care composition according to claim 14, wherein the personal care composition reduces sweat by at least 21%, as measured 1 hour after application of the personal care composition.

17. The personal care composition according to claim 14, wherein the personal care composition reduces sweat by at least 9%, as measured 24 hours after application of the personal care composition.

18. A method of reducing malodor comprising: applying the personal care composition according to claim 1 to a skin surface.

19. The method according to claim 18, wherein the personal care composition is selected from: a stick-type deodorant; a stick-type antiperspirant; a roll-on type deodorant; a roll-on type antiperspirant; a lotion; a cream; and a balm.

20. A method of manufacturing a personal care composition according to claim 1 comprising:
a) mixing methylheptylglycerin, glycerin, xanthan gum, sodium stearate, and glyceryl monolaurate to form a blend;
b) adding a predetermined quantity of the blend to a container; and
c) cooling the blend in the container to room temperature to form a solid state.
